(19) United States
(12) Patent Application Publication
(10) Pub. No.: US 2008/0281083 A1
(43) Pub. Date: Nov. 13, 2008

(54) Antigen of the Pm-2 Antibody and Use Thereof

(75) Inventors: Heinz Vollmers, Wurzburg (DE); Frank Hensel, Wurzburg (DE)

Correspondence Address: PILLSBURY WINTHROP SHAW PITTMAN LLP, ATTENTION: DOCKETING DEPARTMENT, P.O BOX 10500, McLean, VA, 22102, US

(73) Assignee: PATRYS PTY LIMITED, Melbourne, Victoria (AU)

(21) Appl. No.: 10/599,297

(22) PCT Filed: Mar. 10, 2005
(86) PCT No.: PCT/DE2005/000455

§ 371 Date: Nov. 2, 2006

(30) Foreign Application Priority Data
Mar. 25, 2004 (DE) 10 2004 015 179.2

(51) Int. Cl. C07K 14/00; C12N 15/63

(52) U.S. Cl. 530/395; 435/320.1

(57) ABSTRACT

A polypeptide, which is expressed on the cell surface as a membrane-bound protein, is glycolyzed at one or more points (i.e., membrane glycoprotein) and has an amino acid sequence that corresponds partially or completely to that of the integrin binding protein p80 (accession #AJ131720) or REV1 (accession #AF206019.) The membrane glycoprotein, is expressed by neoplastic cells and not by non-neoplastic cells as an antigen, specifically binds the human monoclonal antibody PM-2 (DSM number: DSM ACC2600) and is, in addition, N-glycosidically and O-glucosidically linked. A related method is provided for the isolation or production of the antigen and for the use of the latter for producing a medicament for immunization. The isolated antigen can also be used to identify medicaments with an apoptotic cell-proliferation inhibiting action. The membrane glycoprotein can also be used as a tumor marker.

The invention relates to a polypeptide, which is expressed on the cell surface as a membrane-bound protein, is glycosylated at one or more points (membrane glycoprotein) and whose amino acid sequence corresponds partially or completely to that of the integrin binding protein p80 (accession #AJ131720) or REV1 (accession #AF206019).

The invention also relates to the use of the polypeptide according to the invention in tumour treatment, tumour diagnosis and tumour research.

### BACKGROUND OF THE INVENTION

Despite advances in chemotherapy, successful cancer treatment is currently one of the biggest challenges in medicine. In this aim, early diagnosis of cancer plays a particularly important role. An alarmingly high number of cancer patients are already in an advanced state of illness when the first diagnosis is made. Besides the early detection of tumour cells in the tissue, the search for new means of combating cancer naturally plays a major role, e.g. by inhibition of cell proliferation or by initiating apoptosis. Apoptotic receptors on the cell surface, such as those of the NGF/TNF family are predominantly expressed on lymphocytes, but are also found on various other cell types, and are therefore disadvantageously not suitable for cancer therapy. In particular, in vivo tests ligands and antibodies for these receptors have led to liver damage. Tumour-specific receptors (antigens) with apoptotic function, which are expressed on the surface of neoplastic cells are therefore particularly important for cancer therapy. That is true in particular against the background that human monoclonal antibodies with an apoptotic effect are increasingly being identified and isolated. Through the use of hybridoma technology, there has been success in isolating of a series of tumour-specific IgM antibodies from the tissue of cancer patients and the tissue of healthy donors. In particular, it has already been possible to identify two human monoclonal tumour-specific antibodies and their antigens. Thus, the human monoclonal antibody SC-1 binds specifically to the CD-55 receptor (Cancer Research, 1999 Oct. 15, 59 (20), 5299-5306, Hensel et. al.), while the human monoclonal PAM-1 antibody binds specifically to the CFR-1 receptor (Oncol. Rep. 2004, Apr. 11 (4), 777-784 Brändlein et. al.). Human monoclonal antibodies of this kind are accredited a major role for the treatment and diagnosis of cancer. Their importance for cancer therapy lies in the induction of apoptosis and/or inhibition of cell proliferation after specific binding to the corresponding antigens (receptors) on the surface of neoplastic cells.

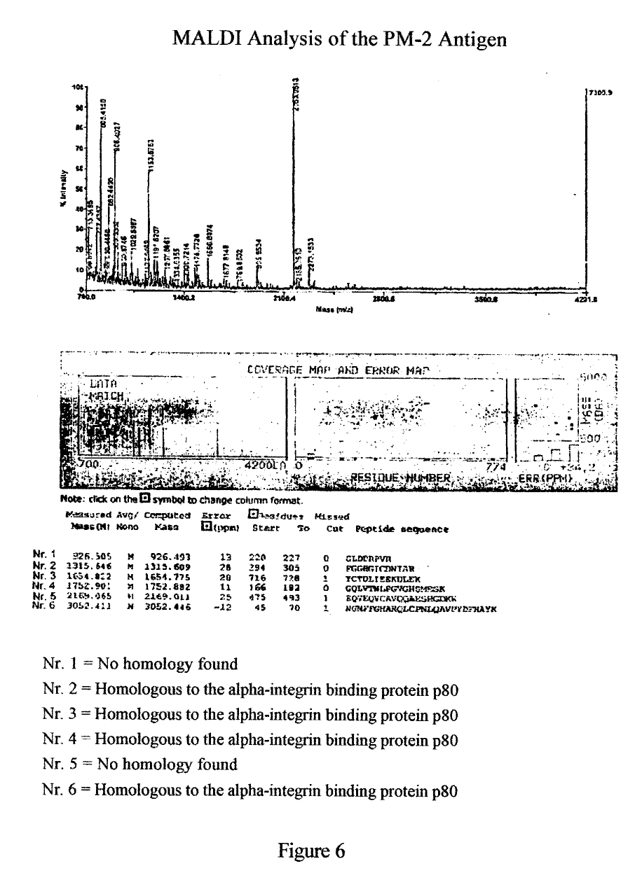

With the aid of the human monoclonal antibody PM-2 (DE 102 305 156 A1) (DSM accession number: DSM ACC2600), it has now been possible to identify the glycomembrane protein according to the invention, which specifically finds the PM-2 antibody as antigen (receptor). A sequence comparison as part of the mass-spectroscopic analysis (see FIG. 6) of the antigen according to the invention disclosed the homology present, at least in the range under investigation, between the antigen according to the invention and a protein known in the prior art (NCBI accession #AJ131720), which is known as the p-80 protein or REV1 (accession #AF206019)

### DEFINITIONS

Apoptosis is programmed cell death, that is to say the suicide of cells by fragmentation of the DNA, cell shrinkage and dilatation of the endoplasmatic reticulum, followed by cell fragmentation and the formation of membrane vesicles, the so-called apoptotic bodies. It is the most frequent cause of death of eukaryotic cells and occurs in embryogenesis, metamorphosis and tissue atrophy. Apoptosis, as the physiological form of cell death, ensures fast and clean removal of unnecessary cells without initiating inflammation processes or tissue injury as in the case of necrosis. Under pathological conditions, apoptosis also serves to remove malignant cells, such as cancer precursor cells. Apoptosis can be initiated by a wide variety of stimuli, such as by cytotoxic T-lymphocytes or cytokines, such as tumour necrosis factor, glycocorticoids and antibodies.

### Glycosylation

Membrane glycoproteins have, on their extracellular side, sugar residues (glycocalyx), which are bound either to the amide nitrogen of an asparagine side chain (N-binding) or to the oxygen atom of a serine or threonine side chain (O-binding). The sugar linked directly to the side chain is usually N-acetylglucosamine or N-acetylgalactosamine. Carbohydrates can form very diverse structures. First, various monosaccharides can be linked together via one or more OH groups. Second, the links attached to the C-1 atom can have an a or a β configuration. Utilising these various bonds, glycomembrane proteins can have extended branches comprised of oligosaccharides.

It is known that the carbohydrate structure (glycosylation pattern, glycocalyx) on the cell surface has an information character for intracellular recognition. Thus, for example, the immune system requires the glycosylation pattern for identification and adsorption to the target cell, though the structural basics for the sequence of this process are not yet understood.

Integrins are proteins, which are coupled to the surface of cells and whose lipophilic part extends through the cell wall (transmembrane proteins) and whose extracellular components are glycosylated (glycomembrane proteins). By a process known as adhesion, integrins promote the binding of cells to the extracellular matrix and to other cells. Besides the amino acid sequence of the integrins and the three-dimensional protein structure, the structures of the sugars bound to the integrins are responsibility for the selectivity of binding. Integrins are heterodimers that are composed of an α and a β subunit, there being about 10 different α-subunits and at least twice as many different β-subunits. The variability growing from this for the receptor type of the integrins alone shows that the general mechanisms underlying cell adhesion are by no means completely understood. The specificity of integrin binding is furthermore modulated by the extracellular Ca²⁺ concentration. It is known that integrins preferentially bind to the Arg-Gly-Asp sequence (Ruoslahti, Pierschbacher, Science, 1987, 238, 491) of the extracellular matrix.

Of the adhesion receptors, integrins operate in particular in signal transduction, that is to say in information transmission of extracellular signals into the interior of the cell and from the interior of the cell to the outside. Adhesion and subsequent signal transmission into the cell interior sets off intracellular processes, which can lead to restructuring of the cytoskeleton and to the induction of signal cascades. Integrin binding proteins are the binding partners of the integrins in adhesion.

Cell adhesion processes have a regulatory effect on the expression pattern and therefore on the specificity of the receptors themselves. Cell-adhesion mechanism are therefore important for cell growth, cell migration and differentiation. In particular, they are involved when cells lose their specialized forms and become metastasing cancer cells.

### Neoplastic Cells

A neoplasma or tumour is an abnormal tissue mass whose growth is autonomous (independent of growth factors), uncoordinated, aimless and progressive. Tumours consist of two components The parenchyma cells, also known as neoplastic cells, and of the non-tumorous stroma, i.e the connecting tissue and blood vessels. A neoplastic cell in the context of the antigen according to the invention designates a cell, which is subject to uncontrolled cell division or a cell that does not have an apoptosis mechanism. A neoplastic cell in the sense of the invention can also have both disorders, and can also be characterized by the fact that its cell cycle departs from the normal cell cycle.

### DESCRIPTION

The sequence (accession #AJ131720) known from the prior art (Wixler et al., FEBS Letters 1999, 445, 351-355) codes for the α-integrin binding protein p80, which interacts with the proximal region of the α-integrin. These binding properties indicate that p-80 must be a membrane-bound protein. No details about the glycosylation of the p-80 protein are known.

The human REV-1 protein (accession #AF206019), which is also known from the prior art, also has, at least in sections, a sequence that is homological to the antigen according to the invention. Deoxycytidyl transferase activity is given as a function of REV1. Deoxycytidyl transferase probably catalyses the binding of desoxycytidylate to the daughter DNA strand during DNA replication in the cell nucleus. REV1, in contrast to the integrin-binding protein p-80, is not a membrane-bound protein and is localised in the nucleus.

The fact that polypeptides with the same amino acid sequence can be present both as membrane-bound proteins and as proteins in the cell nucleus shows that the posttranslational modifications, such as glycosylation plays a major role as regards the localisation and the function of a polypeptide and that despite the homologous sequence, it cannot be assumed that such proteins are identical.

The object of the invention consists in identifying and characterising an antigen to which the tumour-specific human monoclonal antibody PM-2 binds (DSM accessibility number: DSM ACC2600), and in the use of the antigen for tumour treatment and tumour diagnosis.

The PM-2 antibody (German patent DE 102 30 516 A1) is a human monoclonal antibody with heavy and light chain molecules that in each case comprise one region that is constant in structure from antibody to antibody and a region that is variable in structure from antibody to antibody, or a functional fragment thereof, at least one of the variable regions of the light chain comprising substantially that in SEQ. NO. 4 and/or that of the heavy chain in SEQ ID NO. 3 of the sequence protocol. The PM-2 antibody was produced by means of the hybridoma technique, the hybridoma cells (DSM ACC2600) having been obtained by fusion of the hetero-myeloma cells HAB 1 and subclones thereof with P-lymphocytes. The P lymphocytes were taken from a lymphatic organ, preferably the spleen or lymph nodes of a carcinoma patient. The human monoclonal antibody PM-2 is characterised by the fact that, after specific binding to the corresponding PM-2 antigen on the surface of a neoplastic cell, it initiates apoptosis in this cell and/or inhibits cell proliferation. The apoptotic effect of the PM-2 antibody has been proven in detail with the aid of cell death ELISA experiments and is described in detail in document DE 102 30 516 A1.

To achieve this object, the invention teaches a glycomembrane protein with antigen properties, which is characterised in that

it is expressed by neoplastic cells and not by non-neoplastic cells, and,

as an antigen, binds specifically to the human monoclonal antibody PM-2 (DSM accessibility number. DSM ACC2600), and

is N-glycosidically and O-glycosidically glycosylated.

The antigen according to the invention is tumour specific, that is to say it is only expressed by neoplastic cells. For the specific binding of the human monoclonal antibody PM-2 (DSM accessibility number: DSM ACC2600), to the antigen according to the invention, glycosylation is responsible, which is N-glycosidic and O-glycosidic.

Both the antigen according to the invention and the p-80 protein, which is sequence identical at least in sections, are membrane-bound proteins. The fact that the p-80 protein binds integrin provides an indication of the role that the antigen according to the invention plays in the emergence of the tumour. As cell adhesion molecules, integrins are important in angiogenesis. Thus, the αVβ3 integrin is expressed by the endothelials cells of the vessels that supply the tumour. It would be conceivable that the antigen according to the invention, which was detected in the epithelial cells of the blood vessels, also interacts with integrins and its inhibition has a similar effect to that of angiogenesis inhibition. It is also known that integrins play an important role in the metastasis of tumour cells in that they make the adhesion of the tumour cells transported via the blood vessels into hitherto tumour-free tissue possible at all.

For the specific binding of the antibody PM-2, antigens according to the invention, the N-glycosidically bound glycostructures probably play a particular role. The more accurate analysis of the glycosylation sites is performed by means of software known to a person skilled in the art (software of the database of the “UK MRC Human Genome Mapping Project” http://www.hgmp.mrc.ac.uk/GenomeWeb/prot-anal.html).

Starting from the only partially available sequence (774 amino acids) of p-80, this analysis produced the result that the N-glycosylation takes place in particular at the amino acid positions 333, 343, 450 and 568. A corresponding analysis with the completely available sequence of the REV1 proteins produced the result that, in the case of REV1, N-glycosylation takes place in particular at amino acid positions 810, 830, 927 and 1045.

The number of O-glycosylation sites determined by means of the same method is significantly higher.

It lies within the scope of the invention that the antigen identified by means of the human monoclonal antibody is made up of a monomer or of a plurality of identical subunits. The possibility that it is a homomer comprising two identical subunits (dimer) or is associated with other proteins could also explain which the molecular weight determinable by means of immuno blot (Western blot) until now has varied within a wide bandwidth.

The cells expressing the protein have already been mentioned in the context of the characterization of the PM-2 antibody. Reference is therefore made here to document DE 102 305 156 A1. The hybridoma cell line, which produces the antibody PM-2, was deposited on 2 Jul. 2003 under accessibility DSM2600 at the “Deutsche Sammlung von Mikroorganismen und Zellkulturen (DSMZ)” the German Collection of Microorganisms and Cell Cultures, under the provision of the Budapest Treaty for the purposes of patent depositions. For the characterisation of the antigen according to the invention, in particular pancreas carcinoma cells of the cell line BXPC3 (ATCC Number CRL 1687) were used, because the antigen is particularly well expressed on their surface.

For explanation of the immunobiological importance of the antigen according to the invention, it could be decisive that the antigen is expressed on the surface of epithelial cells in the tumour tissue.

Of decisive importance is the therapeutic potential of the antigen according to the invention, which consists in inducing apoptosis in a neoplastic cell after binding of the PM-2 antibody to the antigen. Alternatively, it is conceivable that, because of the specific binding of the PM-2 antibody to the antigen on the surface of neoplastic cells, the cell proliferation thereof is inhibited. Both mechanisms are interesting for tumour therapy.

In the scope of the invention, a process for isolation of the antigen according to the invention was developed. After homogenization and solubilization in a detergent known to the person skilled in the art, the antigen is chromatographically purified. In particular, size-exclusion chromatography is used for this. In an improvement of the isolation method, it is conceivable that size-exclusion chromatography is followed by a further step in the form of anion-exchange chromatography. By means of this second purification step, the purity of the isolated glycomembrane protein is improved.

The antigen isolated in this way can be used for preparing a pharmaceutical using the conventional pharmaceutical excipient and carrier substance. In the simplest case, in-vivo administration of the purified antigen in a physiological NaCl solution is provided.

However, it lies within the scope of the invention that the purified glycomembrane protein is used as an antigen to identify specific binding ligands or adhesion peptides. In principle, it is conceivable that the polypeptides identified in this manner only correspond in sections to the sequence of the human monoclonal antibody, but still initiate apoptosis in neoplastic cells and/or inhibit cell proliferation in these cells. To reinforce this effect, the adhesion peptides or ligands can be coupled to a radionucleotide, a cytotokin, a cytokine, or a growth inhibitor.

It is conceivable to use the glycomembrane protein according to the invention as an antigen in the identification of actives as part of a high-throughput screening. Such methods and developments thereof are known to persons skilled in the art and active in the field of pharmaceutical research.

In the scope of the invention, the use of the antigen according to the invention as a tumour marker is provided. In this case detection of the membrane glycoprotein according to the invention on the surface of neoplastic cells can be performed by means of the PM-2 antibody. By means of the vector insert given in sequence protocols 1 and 2 it was possible to demonstrate in an antisense experiment that the membrane glycoprotein according to the invention is the antigen for the specifically binding human monoclonal antibody PM-2.

### EXAMPLE 1

### Material And Methods

Cell Culture

The carcinoma cell line BXPC-3 (ATCC number CRL1687) was used to obtain the receptor. For comparative studies, e.g. Western blot analysis, the familiar gastric adenioma cell line 23132/87 (DSMZ accessibility number DSM201) (Hensel et. al. 1999, Int. J. Cancer 81:229-235) was used. The cells were grown to 80% confluence in RPMI-1640 (PAA, Vienna, Austria) supplemented with 10% FCS and penicillin/streptomycin (1% for both). For the above-described studies, the cells were removed with trypsin/EDTA and washed twice with phosphate-buffered saline solution (PBS).

Preparation of Membrane Extracts

Isolation of the membrane proteins from tumour cells was performed in the manner described by Hensel et al. (Hensel et al., 1999, Int. J. Cancer 81:229-235), using the cell line BXPC-3. Described briefly, the coherent tumour cells were washed twice with PBS, removed with a cell scraper, centrifuged, and suspended in a hypotonic buffer (20 mM HEPES, 3 mM KCl, 3 mM MgCl₂). After 15 min. incubation on ice and ultrasonic treatment for 5 min., the nuclei were pelletized by centrifuging at 10,000 g for 10 min. The supernatant was centrifuged for 30 min. at 100,000 g in a swing-out rotor and thereby the membrane was pelletized. After the pellets had been washed with the hypotonic buffer, they were suspended again in a membrane lysis buffer (50 mM HEPES pH 7.4, 0.1 mM EDTA, 10% glycerol and 1% Triton X-100). A protease inhibitor (Boehringer, Mannheim, Germany) was added to all solutions.

Purification of the Antigen

Purification of the antigen was carried out with column chromatography using a Pharmazia (Freiburg, Germany) FPLC unit. For size-exclusion chromatography, a Pharmazia Superdex 200 (XK 16/60) column was charged with 5 mg of the membrane preparation and operated with a buffer A (100 mM tris/Cl, pH 7.5, 2 mM EDTA, 40 mM NaCl, 1% Triton X-100). Then the eluate was fractionated and investigated for reactions with the antibody PM-2 by Western blot analysis. The positive fractions were charged on a MonoQ (5/5 column) using buffer A. The bound proteins were washed out by means of a linear gradient using buffer B (100 mM, tris/Cl, pH 7.5, 1 M NaCl, 2 mM EDTA, 1 M NaCl, 1% Triton X-100), fractionated and investigated with Coomassie-stained SDS-PAGE and Western blot analysis.

Western Blotting

Separation by means of 10% SDS-PAGE gels and Western blotting of the proteins was carried out under standard conditions as described elsewhere (Hense et al, 1999, Int J. Cancer 81:229-235). In brief, the blotted nitrocellulose membranes were blocked with PBS, which contained 2% skimmed milk powder, followed by one hour's incubation with 10 pg/ml purified antibody 103/51. After washing three times with PBS +0.05% Tween 20, the second antibody (peroxidase-coupled hare antihuman IgNI antibody (Dianova, Hamburg, Germany) was incubated. The reaction was demonstrated by means of the supersignal chemiluminescence kit from Pierce (KMF, St. Augustin, Germany).

The positive bands identified by means of Western blot on an appropriate SDC gel were separated from the gel and used for MALDI analysis.

MALDI Peptide Analysis

The protein bands separated from the SDS gel were cut into small pieces of about 1 mm×1 mm. The gel pieces were washed, reduced with DTT, s-alkylated with iodoacetamide and treated with trypsin (unmodified, sequence, Boehringer) as described elsewhere (Shevchenko et al., 1996b Anal. Chem. 68:850-858). After 3 hours' digestion at 37° C., 0.3 μl of the solution was removed and subjected to mass spectroscopic analysis (MALIDI) by means of a Bruker Reflex MALDI-TOF, which was equipped with downstream extraction (Brucker, Franzen, Bremen, Germany). The thin-film technique was used for sample preparation (Jensen et al., 1996 Rapid Commun. Mass Spectrom 10:1371-1378). The tryptic peptide masses were used to search for non-redundant protein sequence data by means of a peptide search program that was developed in house.

Cloning of the p80 Antisense Vector And Transfection

RNA isolation, cDNA synthesis and PCR were performed as described (Hensel et al., 1999 In. J. Cancer 81:229-235). In brief, for the amplification of a fragment by means of PCR from a region from position 181 to position 681 of the nucleotide sequence of p-80 (accession #AJ131720), the following primers were used:

P80-Rev3′ 5′ CTGTTCCATACGATTTTCATGC 3′
P80-Rev5′ 5′ TCGAACTGGTCTATCATCCAA 3′

The amplification was carried out with the following cycle profile: 95° C., 2 min; followed by 35 cycles at 94° C., 30 sec; 60° C., 30 sec; 72° C. 60 sec. and subsequently 72° C., 4 min. Cloning in the pCR-Script Amp SK (+) vector and sequencing of the DNA were carried out as described before (Hensel et al., 1999 Int. J. Cancer 81:229-235).

The insert was cut out with appropriate restriction enzymes from the pCR Script Amp SK (+) Vector and subcloned into the pHook-2 Vector (Invitrogen, Leek, Netherlands). Various clones were investigated by sequencing of the successful cloning. A clone was chosen in which the coded sequence had been cloned in the antisense direction to the promoter. This clone was amplified and vectors were isolated for antisense transfection.

Transfection of the cell line BXPC-3 with phook-anti PM-2-R was completed with a prime factor reagent (PQLab, Erlangen, Germany) according to the supplier's manual. To this end, the plasmid DNA was diluted to 10 μg/ml and the prime factor reagent was added to a serum-free growth medium in a ratio of 1:10. The diluted plasmid DNA (450 μl), the diluted prime factor reagent as supplement (90 μl) and the serum-free growth medium (460 μl) were mixed and incubated at room temperature. 60 ml cell culture dishes (70% confluent) were washed twice with the serum-free growth medium and then the prime factor/DNA mixture was added drop by drop. The cells were incubated for 18 hours at 37° C. and 7% CO₂, then the serum-free growth medium was replaced with a growth medium with 10% FCS and the cells were incubated for a further 24 hours before the expression of the receptor protein was investigated.

a) Part of the cell line BXPC-3 was transfixed with a control vector (p-HOOK-2), another part with the p80 antisense vector.

b) 48 h after transfection, Cytospin preparations of the cells were prepared.

c) The cytospin preparations were stained with the PM-2 antibody and a control antibody (only secondary antibody).

d) Cells that have been transfixed with the p-80 antisense vector show a distinct reduction in the binding of the antibody PM-2.

Digestion With N-Glycosidase On Cytospins

The cells used were dissociated from the substrate of their culture bottles by means of trypsin/EDTA and then incubated to regenerate the membrane proteins for 1 h in RPMI-1640 medium+10% FCS at 4° C. Then cytospin preparations were prepared with the cells. The cytospins were dried overnight at RT. After drying, the cells were fixed for 10 min. with 100% acetone and washed three times with PBS. Then the fixed cells were digested with 5 mU/ml N-glycosidase (in 100 μl phosphate buffer, pH 7.0) for 3 hours at 37° C. in the incubator. Then the cytospin preparations were washed three times with PBS and immunohistochemical staining was performed with the different antibodies. As negative control there were used cytospins that had only been incubated with phosphate buffer, or cytospins that had been subjected to normal immunohistochemical staining without glycosidase treatment. Staining was carried out as described. The finished staining was subsequently evaluated microscopically and the results documented with a photographic system and an Olympus microscope.

Digestion With N-Glycosidase On Cytospins

Here, too, the cells were removed with trypsin and reconstituted on ice for 1 h in culture medium. After preparation of the cytospin preparations and subsequent fixing, the cells were incubated with 20 μl/ml O-glyosidase (in 100 μl phosphate buffer, pH 6.8) at 37° C. for 3 h. As a control, cytospins were incubated only with phosphate buffer or normally stained without incubation. Immunohistochemical staining was carried out as described.

Immunohistochemical Staining of Living Cells And Acetone-Fixed Cells

For staining living cells, the cells were dissociated, washed and diluted to 1×10⁶ cells per ml. 1 ml of the cell solution was centrifuged at 1,500 g for 5 min. The antibody diluted to 40 μg/ml with complete RPMI is filled to a final volume of 1 ml and incubated on ice for 90 min. Then the cells are pelletized at 1,500 g for 5 min. and dissociated again with 500 μl RPMI. The cytospin preparations are prepared with 200 μl of the cell solution, and air dried for 30 min. The cells are fixed in acetone for 30 min. and washed three times with Tris/NaCl. The HRP-coupled hare antihuman IgM (DAKO) are diluted 1:50 in PBS/BSA (0.1%) and incubated for 30 min. at room temperature. After washing three times, staining is carried out as mentioned above.

For staining the acetone-fixed cells, the cytospins are prepared, air dried at room temperature and, as described above, fixed in acetone. Then the cytospins are blocked for 15 min. with PBS/BSA (0.1%) and incubated for 30 min. with 10 μg/ml primary antibodies and then washed three times. Incubation with the secondary antibodies and staining are carried out as described above.

### EXAMPLE 2

### Result of Glycosidase—Digestion

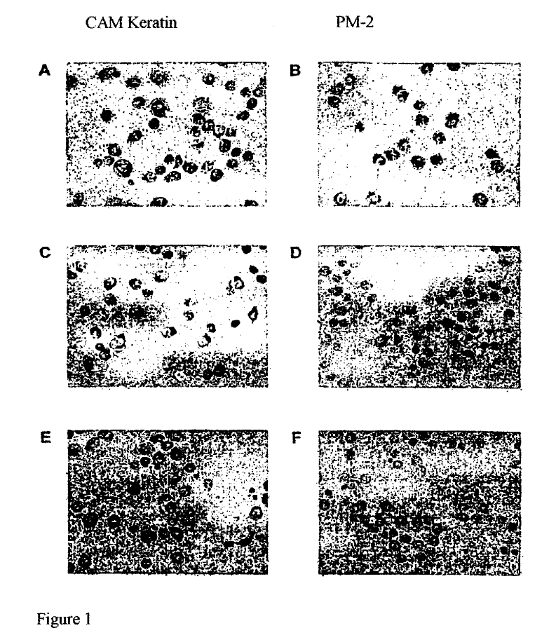

FIG. 1 shows the influence of glycosidase digestion on the antibody binding of PM-2 to the cell surface of the pancreas carcinoma cells BXPC-3. After digestion, the cytospins were immunohistochemically stained with the positive control CAM Keratin (A, C, E) and with PM-2 (B, D, F).

Figures A and B in FIG. 1 show the controls after incubation of the cells in glycosidase buffer without enzyme. Figures C and D show the effects of N-glycosidase incubation on the binding of the antibody PM-2 to the pancreas carcinoma cells. After digestion with the enzyme N-glycosidase can no longer be stained with the antibody PM-2. This means that the antibody can no longer bind to its receptor, because the bound glycostructure necessary for the specific binding was cleaved off during N-glycosidase digestion.

The figures in FIG. 1 E and F show the effects of O-glycosidase incubation on the binding of the antibody PM-2. While the positive control, CAM keratin in Fig. E does not show a changed colour, after digestion with the O-glycosidase enzyme it is found that the cells can no longer be stained with the antibody PM-2. That suggests that, besides the N-bound sugar, at least also an O-glycosidically bound determinant of the antigen was responsible for the specific binding of the PM-2 antibody.

### EXAMPLE 3

### Result of Antisense Transfection

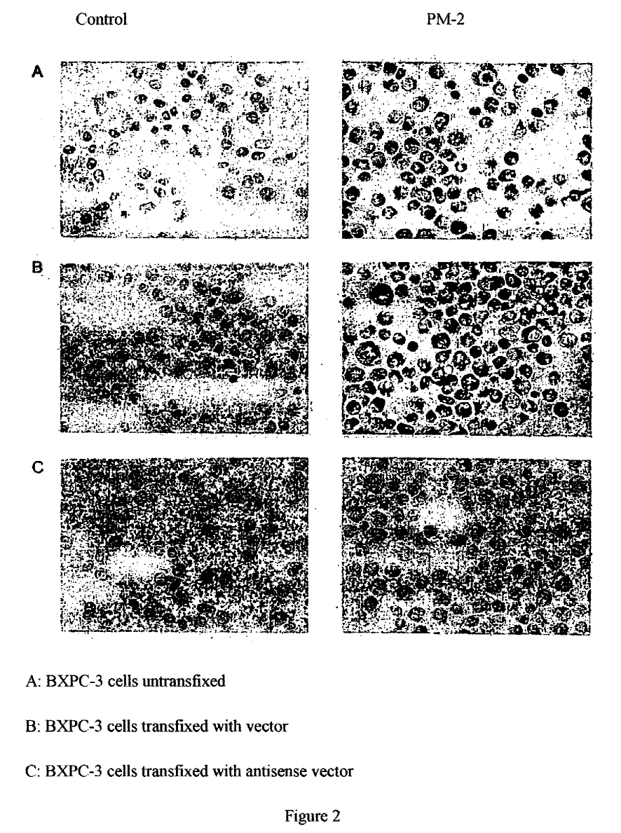

FIG. 2 shows the effect of antisense transfection on stainings with antibodies PM-2 and living cell staining (200× enlargement).

The right-hand column of FIG. 2 show cells of the BXPC-3 cell line that have been stained with PM-2. The tipper row shows non-transfixed cells. The centre row shows the cells transfixed with the empty vector. In both cases, the cells show a distinct PM-2 antibody staining. This staining decreases significantly after transfection of the cells with the antisense vector. This shows the image in the right-hand column of the lower row. This experiment shows that the PM-2 antibody binds to a membrane protein whose amino acid sequence whose amino acid sequence must be at least in sections homologous to the amino acid sequence of the P-80 protein.

Together with the result of the glucosidase digestion, this shows that a glycomembrane protein whose amino acid sequence corresponds at least partly to that of the P-80 protein is the antigen to which the PM-2 antibody specifically binds.

### EXAMPLE 3

### Result of Western Blot

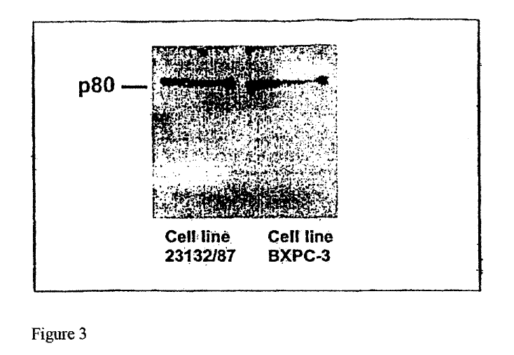

FIG. 3 shows the immunospecific evidence of the antigen expressed in BXPC-3 cells and in 23132/87 cells with the aid of the PM-2 antibody.

### EXAMPLE 4

### Determination of the N-Glycosilation Sites

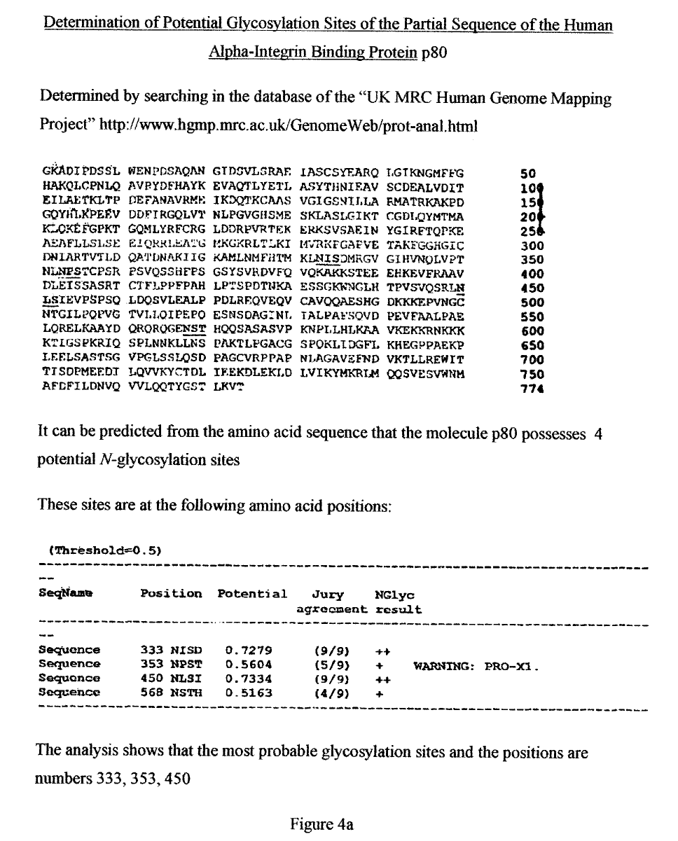

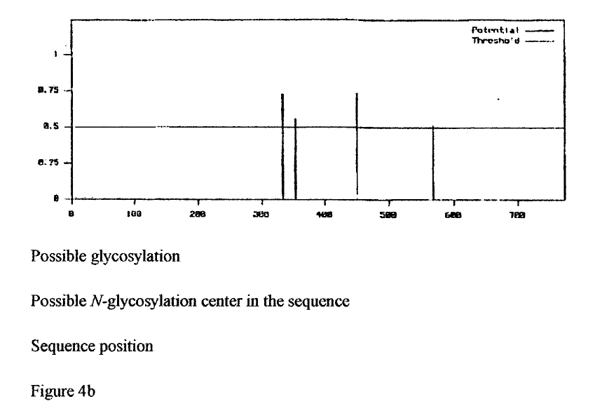

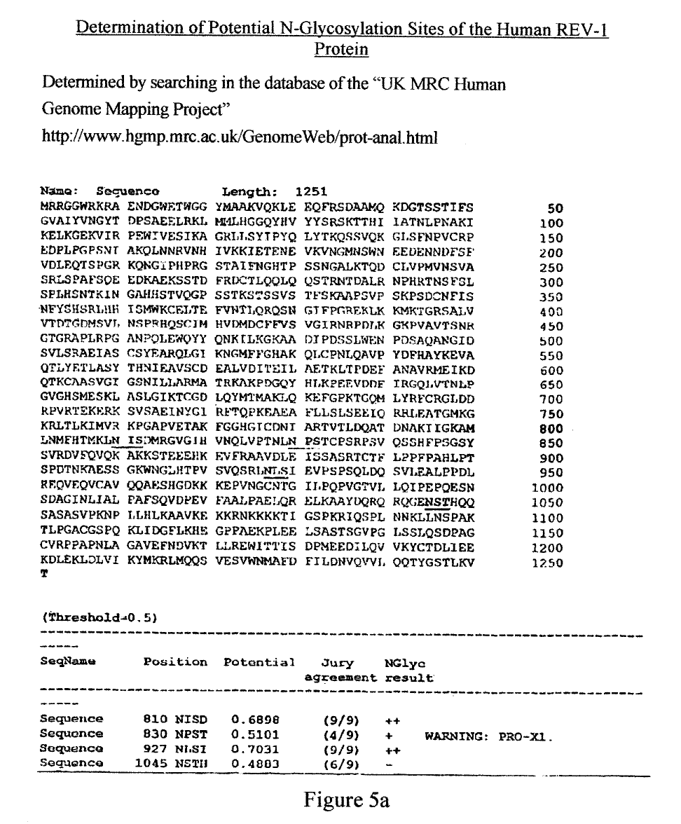

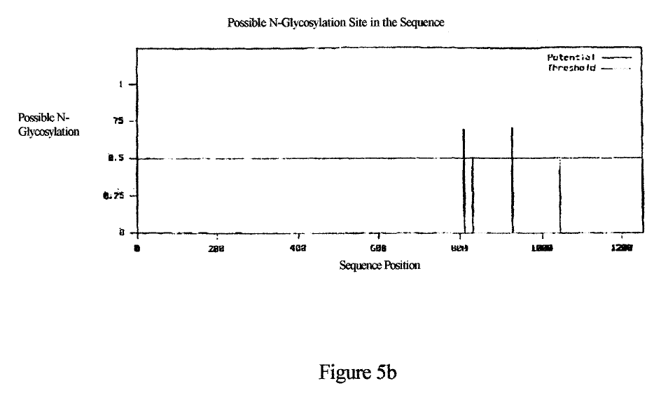

The glycosilyation sites indicated in FIGS. 4a and 4b and 5a and 5b were determined with the aid of the software of the database of the “UK MRC Human Genome Mapping Project” (http://www.hgmp.mrc.ac.uk/GenomeWeb/protanal.html).

### EXAMPLE 5

### MALDI Analysis of the PM-2 Antigen

FIG. 6 shows the result of the mass-spectroscopic analysis of protein bands selected from an SDS gel with the aid of the PM-2 antibody. A sequence comparison of the peptide sections No. 2, No. 3, No. 4 and No. 6 determined with the aid of the mass spectrometer shows sequence homology with the p-80 protein or with the REV1 protein.

1-21. (canceled)

22. A polypeptide expressed on a cell surface as a membrane-bound protein is glycosylated at one or more points, forming a membrane glycoprotein, and has an amino acid sequence corresponding partially or completely to that of an integrin binding protein p80 (accession #AJ131720) or REV1 (accession #AF206019), wherein said membrane glycoprotein is expressed by neoplastic cells, and not by non-neoplastic cells, and, as an antigen, binds specifically to a human monoclonal antibody PM-2 (DSM accessibility number: DSM ACC2600), and is N-glycosidically and O-glycosidically glycosylated.

23. The polypeptide according to claim 22, wherein specific binding of the human monoclonal antibody PM-2 (DSM accessibility number: DSM ACC2600) to the membrane glycoprotein is mediated by at least one N-glycosylated carbohydrate residue.

24. The polypeptide according to claim 22, wherein an N-glycosylation is present on at least one of the amino acid positions 333, 353, 450 and 568 of the partial sequence (accession #AJ131720) of the p-80 antigen.

25. The polypeptide according to claim 22, wherein an N-glycosilyation is present on at least one of the amino acid positions 810, 830, 927 and 1045 of the partial sequence (accession #AF206019) of the REV1 antigen.

26. The polypeptide according to claim 22, wherein the membrane glycoprotein is a monomer or is constructed from at least one identical subunit or is associated with other proteins.

27. The polypeptide according to claim 22, wherein the membrane glycoprotein has a molecular weight of approximately 80 to 160 kDa.

28. The polypeptide according to claim 22, wherein the membrane glycoprotein is expressed by the neoplastic cells of the:
large intestine;
pancreas;
the prostate;
the womb, the Fallopian tube;
adrenal gland;
the lung, and by the Squamous cell carcinoma of the oesophagus or the lung;
the gastric carcinoma; and
the ductal carcinoma of the breast,
and not by non-neoplastic cells.

29. The polypeptide according to claim 22, wherein, after binding of the PM-2 antibody (DSM accessibility number: DSM ACC2600) to the membrane glycoprotein on a neoplastic cell, apoptosis being induced in said neoplastic cell and not in non-neoplastic cells.

30. The polypeptide according to claim 22, wherein the membrane glycoprotein is expressed on the surface of the pancreas carcinoma cell line BXPC-3 (ATCC number CRL1687).

31. The polypeptide according to claim 22, wherein the membrane glycoprotein is expressed on the surface of neoplastic epithelium cells.

32. The polypeptide according to claim 22, wherein, after binding of the PM-2 antibody (DSM accessibility number: DSM ACC2600) to a neoplastic cell, cell proliferation is inhibited via the membrane glycoprotein in the cell.

33. A method for obtaining a polypeptide expressed on a cell surface as a membrane-bound protein, said polypeptide, being glycosylated at one or more points, forming a membrane glycoprotein, and having an amino acid sequence corresponding partially or completely to that of an integrin binding protein p80 (accession #AJ131720) or REV1 (accession #AF206019), wherein said membrane glycoprotein is expressed by neoplastic cells, and not by non-neoplastic cells, and as an antigen binds specifically to a human monoclonal antibody PM-2 (DSM accessibility number: DSM ACC2600), and is N-glycosidically and O-glycosidically glycosylated, said method for obtaining said polypeptide comprising the step:
isolating said membrane glycoprotein from membrane extracts of a membrane extracts of a pancreas carcinoma cell line BXPC-3.

34. The method for obtaining a polypeptide expressed on a cell surface as a membrane-bound protein according to claim 33, wherein said step of isolating said membrane glycoprotein includes the steps of:
solubilization of said membrane extracts of said pancreas carcinoma cell line BXPC-3; and,
size-exclusion chromatography of a product obtained from said solubilization step.

35. The method for obtaining a polypeptide expressed on a cell surface as a membrane-bound protein according to claim 34, further comprising the step of:
anion-exchange chromatograph of a product obtained from said step of size-exclusion chromatography.

36. An antisense vector comprising an amino acid sequence of which a portion of said amino acid sequence is indicated in SEQ NO ID:1.

37. An antisense vector comprising a nucleotide sequence of which a portion of said nucleotide sequence is indicated in SEQ NO ID:2.


SEQUENCE LISTING (4 sequences)

SEQ ID NO: 1 - protein, 774 aa, Homo sapiens
  misc_feature: Human alpa-integrin binding protein p80
GKADIPDSSLWENPDSAQANGIDSVLSRAEIASCSYEARQLGIKNGMFFGHAKQLCPNLQAVPYDFHAYKEVAQTLYETLASYTHNIEAVSCDEALVDITEILAETKLTPDEFANAVRMEIKDQTKCAASVGIGSNILLARMATRKAKPDGQYHLKPEEVDDFIRGQLVTNLPGVGHSMESKLASLGIKTCGDLQYMTMAKLQKEFGPKTGQMLYRFCRGLDDRPVRTEKERKSVSAEINYGIRFTQPKEAEAFLLSLSEEIQRRLEATGMKGKRLTLKIMVRKPGAPVETAKFGGHGICDNIARTVTLDQATDNAKIIGKAMLNMFHTMKLNISDMRGVGIHVNQLVPTNLNPSTCPSRPSVQSSHFPSGSYSVRDVFQVQKAKKSTEEEHKEVFRAAVDLEISSASRTCTFLPPFPAHLPTSPDTNKAESSGKWNGLHTPVSVQSRLNLSIEVPSPSQLDQSVLEALPPDLREQVEQVCAVQQAESHGDKKKEPVNGCNTGILPQPVGTVLLQIPEPQESNSDAGINLIALPAFSQVDPEVFAALPAELQRELKAAYDQRQRQGENSTHQQSASASVPKNPLLHLKAAVKEKKRNKKKKTIGSPKRIQSPLNNKLLNSPAKTLPGACGSPQKLIDGFLKHEGPPAEKPLEELSASTSGVPGLSSLQSDPAGCVRPPAPNLAGAVEFNDVKTLLREWITTISDPMEEDILQVVKYCTDLIEEKDLEKLDLVIKYMKRLMQQSVESVWNMAFDFILDNVQVVLQQTYGSTLKVT

SEQ ID NO: 2 - protein, 1251 aa, Homo sapiens
  misc_feature: Human REV-1 prrotein
MRRGGWRKRAENDGWETWGGYMAAKVQKLEEQFRSDAAMQKDGTSSTIFSGVAIYVNGYTDPSAEELRKLMMLHGGQYHVYYSRSKTTHIIATNLPNAKIKELKGEKVIRPEWIVESIKAGRLLSYIPYQLYTKQSSVQKGLSFNPVCRPEDPLPGPSNIAKQLNNRVNHIVKKIETENEVKVNGMNSWNEEDENNDFSFVDLEQTSPGRKQNGIPHPRGSTAIFNGHTPSSNGALKTQDCLVPMVNSVASRLSPAFSQEEDKAEKSSTDFRDCTLQQLQQSTRNTDALRNPHRTNSFSLSPLHSNTKINGAHHSTVQGPSSTKSTSSVSTFSKAAPSVPSKPSDCNFISNFYSHSRLHHISMWKCELTEFVNTLQRQSNGIFPGREKLKKMKTGRSALVVTDTGDMSVLNSPRHQSCIMHVDMDCFFVSVGIRNRPDLKGKPVAVTSNRGTGRAPLRPGANPQLEWQYYQNKILKGKAADIPDSSLWENPDSAQANGIDSVLSRAEIASCSYEARQLGIKNGMFFGHAKQLCPNLQAVPYDFHAYKEVAQTLYETLASYTHNIEAVSCDEALVDITEILAETKLTPDEFANAVRMEIKDQTKCAASVGIGSNILLARMATRKAKPDGQYHLKPEEVDDFIRGQLVTNLPGVGHSMESKLASLGIKTCGDLQYMTMAKLQKEFGPKTGQMLYRFCRGLDDRPVRTEKERKSVSAEINYGIRFTQPKEAEAFLLSLSEEIQRRLEATGMKGKRLTLKIMVRKPGAPVETAKFGGHGICDNIARTVTLDQATDNAKIIGKAMLNMFHTMKLNISDMRGVGIHVNQLVPTNLNPSTCPSRPSVQSSHFPSGSYSVRDVFQVQKAKKSTEEEHKEVFRAAVDLEISSASRTCTFLPPFPAHLPTSPDTNKAESSGKWNGLHTPVSVQSRLNLSIEVPSPSQLDQSVLEALPPDLREQVEQVCAVQQAESHGDKKKEPVNGCNTGILPQPVGTVLLQIPEPQESNSDAGINLIALPAFSQVDPEVFAALPAELQRELKAAYDQRQRQGENSTHQQSASASVPKNPLLHLKAAVKEKKRNKKKKTIGSPKRIQSPLNNKLLNSPAKTLPGACGSPQKLIDGFLKHEGPPAEKPLEELSASTSGVPGLSSLQSDPAGCVRPPAPNLAGAVEFNDVKTLLREWITTISDPMEEDILQVVKYCTDLIEEKDLEKLDLVIKYMKRLMQQSVESVWNMAFDFILDNVQVVLQQTYGSTLKVT

SEQ ID NO: 3 - DNA, 22 bp, Homo sapiens
  misc_feature: P80-Rev 3′
ctgttccatacgattttcatgc

SEQ ID NO: 4 - DNA, 21 bp, Homo sapiens
  misc_feature: P80-Rev 5′
tcgaactggtctatcatccaa